(19) United States
(12) Patent Application Publication
(10) Pub. No.: US 2015/0217098 A1
(43) Pub. Date: Aug. 6, 2015

(54) THERAPEUTIC ELASTIC TAPE

(71) Applicant: Matthew HICKEN, (US)

(72) Inventors: Matthew Hicken, Frisco, TX (US); Steven Silvey, Dallas, TX (US); Gregory Hicken, Logan, UT (US); Christian Hicken, Dallas, TX (US); Nicholas Hicken, Frisco, TX (US)

(21) Appl. No.: 14/421,789

(22) PCT Filed: Aug. 12, 2013
(86) PCT No.: PCT/US13/54559

§ 371 Date: Feb. 13, 2015

Related U.S. Application Data
(60) Provisional application No. 61/682,670, filed on Aug. 13, 2012.

(51) Int. Cl. A61M 35/00; B05D 7/00; B05D 5/00; A61F 7/03; A61F 5/01

(52) U.S. Cl. CPC A61M 35/00; A61F 7/03; A61F 5/0102; B05D 5/00; B05D 7/00; A61F 2007/0226

(57) ABSTRACT

A system, method, and therapeutic elastic tape. The therapeutic elastic tape includes an elastic fabric having a back side and a face side, an adhesive material applied to the elastic fabric. The therapeutic elastic tape further includes a medication applied to at least the back side of the elastic fabric for diffusion to a portion of a body of the user during utilization.

### BACKGROUND

Musculature injuries have increased significantly in recent years. More and more individuals are participating in exercises, competitions, sports, and other indoor and outdoor activities. In addition, many children are starting these activities earlier and many adults are continuing to participate in these activities much later in life resulting in even more injuries.

In some cases, athletic tape is utilized by individuals or medical professionals to support muscle injuries and provide needed support to joints. Development of athletic tape has not been fully implemented to support practice, performance, and recovery of individuals.

### SUMMARY

One embodiment provides a system, method, and therapeutic elastic tape. The therapeutic elastic tape may include an elastic fabric having a back side and a face side, an adhesive material applied to the elastic fabric. The therapeutic elastic tape may further include a medication applied to at least the back side of the elastic fabric for diffusion to a portion of a body of the user during utilization.

Another embodiment provides a method of manufacturing therapeutic elastic tape. An elastic fabric may be combined to have a back side and a face side. An adhesive material may be applied to the back side of the fabric. A medication may be applied to at least the back side of the fabric for absorption by a user when the therapeutic elastic tape is applied.

Yet another embodiment includes a therapeutic elastic tape. The therapeutic elastic tape may include an elastic fabric having a back side and a face side. The therapeutic elastic tape may further include an adhesive material applied to the back side of the elastic fabric. The adhesive material may include a medication for absorption by into a body of a user when the therapeutic elastic tape is applied to the user.

### BRIEF DESCRIPTION OF THE DRAWINGS

Illustrative embodiments of the present invention are described in detail below with reference to the attached drawing figures, which are incorporated by reference herein and wherein:

FIGS. 1A-1C show a bottom view of therapeutic elastic tape in accordance with illustrative embodiments;

FIG. 2 is a spreadsheet of medications for therapeutic elastic tape in accordance with an illustrative embodiment;

FIG. 3 is a pictorial representation of therapeutic elastic tape for a wrist of a user in accordance with an illustrative embodiment;

FIG. 4 is a pictorial representation of therapeutic elastic tape for a shoulder of a user in accordance with an illustrative embodiment;

FIG. 5 is a pictorial representation of therapeutic elastic tape for ribs of a user in accordance with an illustrative embodiment;

FIG. 6 is a pictorial representation of therapeutic elastic tape for a neck of a user in accordance with an illustrative embodiment;

FIG. 7 is a pictorial representation of therapeutic elastic tape for a lower back of a user in accordance with an illustrative embodiment;

FIG. 8 is a pictorial representation of therapeutic elastic tape for a knee of a user in accordance with an illustrative embodiment;

FIG. 9 is a pictorial representation of therapeutic elastic tape for a hip of a user in accordance with an illustrative embodiment;

FIG. 10 is a pictorial representation of therapeutic elastic tape for an elbow of a user in accordance with an illustrative embodiment;

FIG. 11 is a pictorial representation of therapeutic elastic tape for an ankle of a user in accordance with an illustrative embodiment;

FIG. 12 is a pictorial representation of therapeutic elastic tape for a quadricep of a user in accordance with an illustrative embodiment;

FIG. 13 is a pictorial representation of therapeutic elastic tape for treating plantar fasciitis of a user in accordance with an illustrative embodiment;

FIG. 14 is a pictorial representation of therapeutic elastic tape for treating a chest of a user in accordance with an illustrative embodiment;

FIG. 15 is a pictorial representation of therapeutic elastic tape for treating a mid-back of a user in accordance with an illustrative embodiment;

FIG. 16 is a pictorial representation of therapeutic elastic tape for treating a hamstring of a user in accordance with an illustrative embodiment;

FIG. 17 is a pictorial representation of therapeutic elastic tape for treating a forearm of a user in accordance with an illustrative embodiment;

FIG. 18 is a pictorial representation of therapeutic elastic tape for treating a calf of a user in accordance with an illustrative embodiment; and

FIG. 19 is a flowchart of a process for manufacturing therapeutic elastic tape in accordance with an illustrative embodiment.

### DETAILED DESCRIPTION OF THE DRAWINGS

The illustrative embodiments provide various embodiments of therapeutic elastic tape, a method of use, and a method of manufacture. The therapeutic elastic tape is a multi-purpose externally applied tape (supra-cutaneous) that may that may concomitantly facilitate and inhibit musculoskeletal, neuromuscular, and physiological system processes. The therapeutic elastic tape may adhere to or be attached to the user (or patient) or be configured to wrap around limbs or a portion of the user's body.

In one embodiment, the therapeutic elastic tape is a medicated adhesive elastic therapeutic tape. For example, the therapeutic elastic tape may be medicated kinesiology tape (also known as “kinesio tape”). The therapeutic elastic tape may be utilized by individual users as well as healthcare professionals. The therapeutic elastic tape is designed for ease of self-application. However, the medicated tape may also be configured in non-stretch embodiments based on the fabrics, threads, and integrated materials and applications. For example, wrapping applications and specific body parts, support, or rehabilitation of injuries may require medicated tape that does not stretch.

In one embodiment, the therapeutic elastic tape may be infused with, integrated with, or otherwise include any number of medicines, nutrients, ergogenic aides, transport and delivery agents, vitamins, supplements, herbs, chemicals, or medicinal combinations (“medications” or “treatment”). In one embodiment, the therapeutic elastic tape may include prescription drugs, such as pain killers, steroids, or anti-inflammatories that may only be prescribed by a doctor. The medications may be infused, injected, permeated, printed, coated, sprayed, soaked, baked, seared, or otherwise attached or integrated with the elastic tape. The medications may be in liquid, gel, powder, capsule, crystalline, or other forms for absorption into and through the tissues and organs of the body.

The therapeutic elastic tape may have the ability to practice, performance, and recovery for athletes as well as enhance, treat, rehabilitate, and stabilize musculoskeletal and neurological deficiencies. The medicated kinesio tape may also be utilized to prevent injuries and provided needed support and medical treatments. The medications may facilitate motion (e.g. training, competitions, etc) as well as enhance recovery. The therapeutic elastic tape may also be utilized to deliver medications when other more traditional delivery techniques are undesirable, contraindicated, or unavailable. The therapeutic elastic tape may provide targeted topical delivery that may correspond to various forms of treatment (e.g. heat, solar components, convection, motion, cold, electric currents, utilization) that may drive the need for the treatment(s) delivered by the therapeutic elastic tape. In other embodiments, the different forms of treatment may work in tandem. For example the therapeutic elastic tape may include electrodes for applying an electrical current, before, during or after a specified activity.

In one embodiment, the therapeutic elastic tape is a medicated adhesive tape impregnated or infused with analgesic, anti-inflammatory, counter-irritants, electrical current, anesthetics, heat/cold, or a combination of treatments. In addition, any number of traditional or non-traditional herbs, lotions, ointments, creams, oils, remedies, or other medicines may be integrated with the therapeutic elastic tape. The medications may be integrated with, impregnated within, diffused throughout, sprayed on, or otherwise part of the adhesives, the body of the tape, or both. The medicines may diffuse across the skin into skin, muscles, tendons, ligaments and joints from the active tape adhesive.

In one embodiment, the medications are activated for delivery by application, heat/cold, electricity, perspiration, manipulation or mechanical motion, chemical activation, or motion. As a result, as the user moves the portion of the body to which the medicated adhesive tape is secured, the medications may be delivered. The medication within the tape adhesive may thereby be diffused transdermally. In addition, the therapeutic elastic tape may be employed to facilitate, mechanically correct, or inhibit musculoskeletal function while simultaneously serving as the stability base for the medicated adhesive. The deliverable treatment components may be bound to medicated delivery/transport agents or binders for ensuring the immediate, motion activated, time-based, prolonged, or ongoing delivery of the treatments. For example, layers of medication may be configured to be absorbed or delivered at known delivery rates for providing the treatment through an entire event, game, competition, rehabilitation session, day, span of days, or other time period. The delivery may be controlled through distinct layers of fabric infused with medical treatments, structured drugs (e.g. dots, strips, or so forth included with time-based binding components), cold/heat, electricity, perspiration, chemical reaction or so forth.

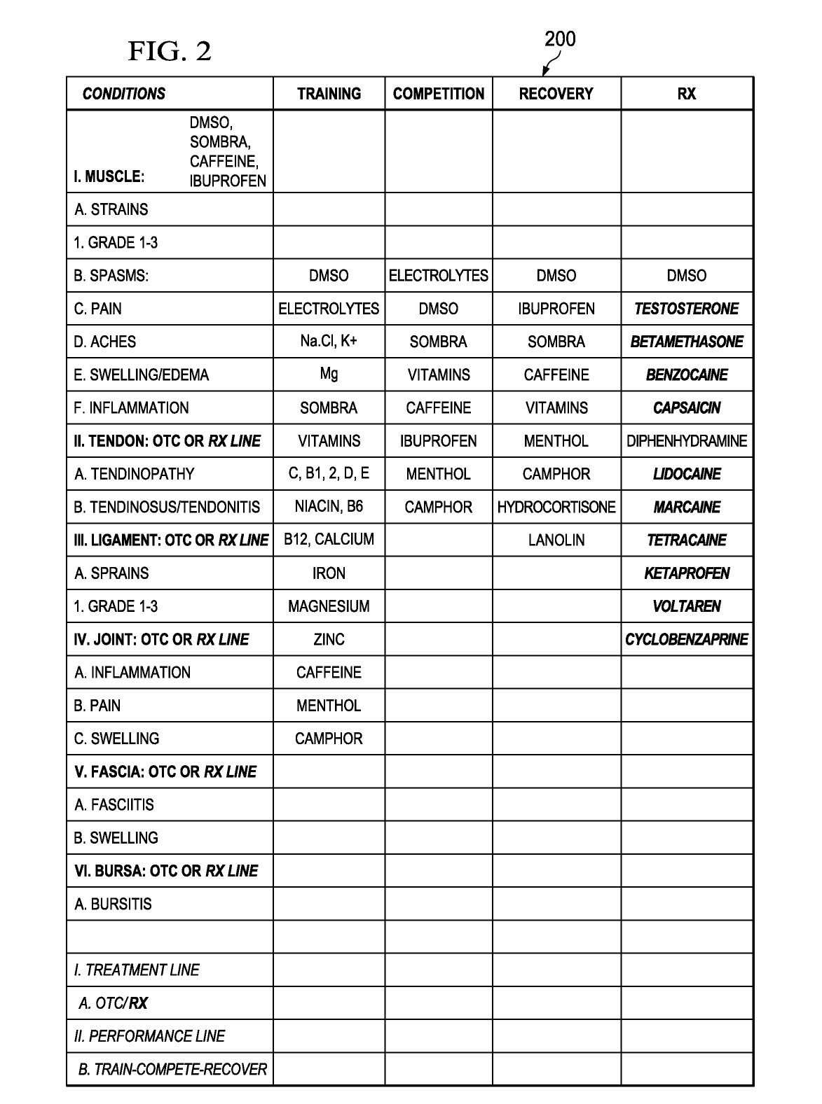

For example, if an athlete wants to facilitate hamstring awareness during practice the athlete may apply the therapeutic elastic tape (see FIG. 2 for treatments) over the hamstrings from origin to insertion. Appropriately, if the athlete wishes to further augment hamstring support during performance, the user may apply the therapeutic elastic tape (see FIG. 2 for specific therapeutic elastic tape combinations for competition). During the post-training or recovery phase the athlete may wish to decrease inflammation, pain, and edema. As a result, the user may appropriately apply therapeutic elastic tape including specified medicines for enhancing recovery. In one embodiment, athletes needing additional treatment options may be able to obtain therapeutic elastic tape infused with prescription medications.

In one embodiment, the therapeutic elastic tape may include multiple layers, such as a outside or external layer (visible when worn), an impermeable layer or agent layer for preventing the treatment from diffusing the outside layer, and one or more delivery layers in contact with the body of the user for both providing support and delivering the medication/treatments. The therapeutic elastic tape may also include perspiration channels to divert perspiration and retain effectiveness of the treated adhesive. The delivery layers may also include an adhesive layer for binding or attaching the therapeutic elastic tape to the user.

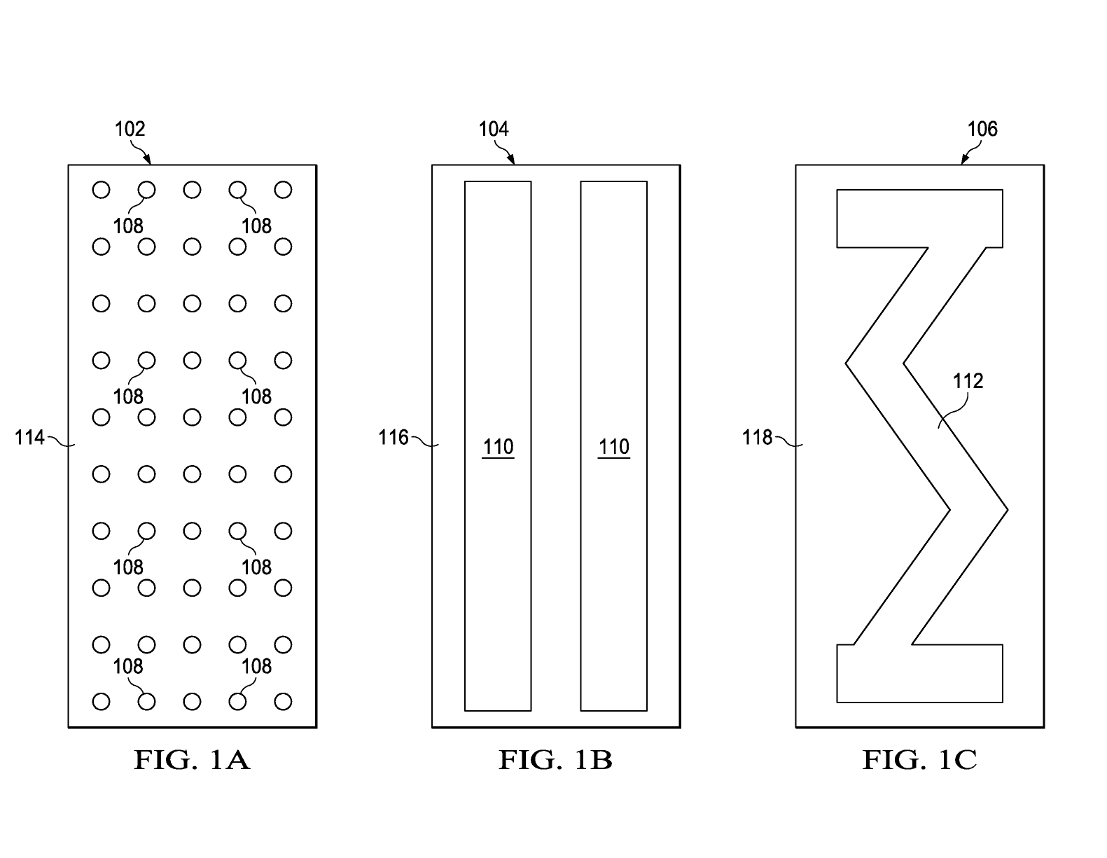

FIGS. 1A-1C show a bottom view of therapeutic elastic tape in accordance with illustrative embodiments. In one embodiment, medications are integrated with the adhesive of the therapeutic elastic tape. As a result, the medications are delivered at every point where the adhesives are in contact with the user. The medications and adhesives may be mixed during manufacturing. The medicated adhesives may then be applied to one or more sides of the therapeutic elastic tape. In one embodiment, the adhesive is sprayed, injected, or diffused onto a first or back side of the therapeutic elastic tape shown by the bottom view of FIGS. 1A-1C. For example, the adhesive may be silicones, acrylics, polyisobutylenes, epoxies, styrene block co-polymers, and other bio adhesives, pressure-sensitive adhesives, or combinations thereof, such as those manufactured by Dow Corning®, Scapa, Ethicon (i.e. Dermabond products produced by Johnson and Johnson). In some embodiments, the adhesive may be a gel, liquid, solid, film, or combination thereof. The adhesive may also utilize developing adhesive formulations, such as hydrocolloid, hydrophilic, or conductive adhesives. For example, hydrophilic and hydrophobic tapes and films are moisture vapor permeable while also capable of providing a seal against liquids. Electrically conductive adhesives may provide high, sustained adhesion with no residue on removal.

In one embodiment, the medicated adhesive may be applied to an entire (user facing or contacting) layer or portion of the therapeutic elastic tape or in a pattern, points, or so forth. The front side or face side of the therapeutic elastic tape may be water resistant, include designs, support structures (e.g. plastic supports, multiple layers, etc.), solar cells, or be otherwise configured.

The therapeutic elastic tape may include an impermeable layer or release agent or a waterproof layer configured to prevent the mixture from spreading throughout the entire portion of the therapeutic elastic tape. The impermeable layer may prevent the medications and adhesives from spreading to the top surface of face side of the therapeutic elastic tape. For example, the impermeable layer may be injected into a middle portion of the therapeutic elastic tape. In another embodiment, a body of the therapeutic elastic tape may have different layers corresponding to an outer layer, impermeable layer, and medicated adhesive layer. In one embodiment, each layer is configured to stretch or deform significantly while still delivering medications. For example, the outer layer may be formed of urethane for deforming to fit the body of the user during motion.

The layers may be secured together by material (e.g. sewing or threads), adhesives, lamination, thermal or cryo-bonding, compression, mechanical components (e.g. clips, hook and latch, etc) or a combination thereof. However, the therapeutic elastic tape may have any number of layers suitable for securing the therapeutic elastic tape for delivery to the user. In another embodiment, the therapeutic elastic tape may not utilized medicated adhesive.

In one embodiment, the adhesive or medicated layer may be covered with a backing layer that may be removed to apply the therapeutic elastic tape to the user. The backing layer may prevent the adhesives and medication from being contaminated, oxidizing, or losing effectiveness before being applied.

FIGS. 1A-1C show therapeutic elastic tape 102, 104, and 106. The therapeutic elastic tape 102, 104, and 106 may each have a pattern 108, 110, and 112. In one embodiment, the patterns 108, 110, and 112 may be composed of medications as are herein described. The free spaces 114, 116, and 118 in each of the respective therapeutic elastic tapes 102, 104, and 106 may represent adhesives for securing the therapeutic elastic tapes 102, 104, and 106 to a portion of the user's body.

In one embodiment, the therapeutic elastic tape may include a core, support, or outer layer that may be formed of fabric (e.g. raw wool fibers, linen, cotton, etc.). In one embodiment, the therapeutic elastic tape may be able to stretch to 120-150% of its original length or 30-50% of its resting longitudinal length. Materials such as cotton may allow for good evaporation allowing the therapeutic elastic tape to remain positioned on the body for a greater amount of time. The fabric may be any number of natural or artificial materials and strands. The core layer formed by weaving, knitting, crocheting, knotting, or pressing fibers together. In one embodiment, the core layer includes elastic components to ensure that the core layer may stretch along with the other layers of the therapeutic elastic tape 102, 104, and 106. The adhesives utilized are configured to be adhered to the skin without irritating the user's skin. Any combination of materials may be selected to adhere the therapeutic elastic tape to the user. In addition, the packaging or the therapeutic elastic tape itself may include a printed label indicating the adhesive, medication, and material composition that may allow user's to avoid known allergic reactions or conditions that may be aggravated by the therapeutic elastic tape. For example, the adhesive may include polycrylate or ethyl acetate. In one embodiment, the therapeutic elastic tape 102, 104, and 106 may be configured to stretch or elongate in one direction, but not in another. For example, the therapeutic elastic tapes may stretch laterally, but be stiff longitudinally or the therapeutic elastic tapes 102, 104, and 106 may stretch longitudinally and be fixed laterally. In addition, one or more sections may be configured to be dynamic/elastic or static/non-elastic.

In another embodiment, the patterns 108, 110, and 112 may represent adhesives and the free spaces 114, 116, and 118 may represent the medications. The patterns 108, 110, and 112 may also represent medicated adhesives or non-medicated adhesives as described herein. The patterns 108, 110, and 112 are also applicable to the specific shapes and configurations of tape as are further described in the subsequent description.

The therapeutic elastic tape 102 may include the pattern 108. In one embodiment, the pattern 108 may include medicated dots that protrude from an interior surface of the therapeutic elastic tape 102. In another embodiment, the pattern 108 may represent medicines diffused (do not protrude or extend) within the therapeutic elastic tape 102. The pattern 108 may ensure that the medications are evenly distributed over a portion of the user's body. In other embodiments, the therapeutic elastic tape 102 may include protuberances, ridges, channels, circular or line patterns extending perpendicularly from the plane of the therapeutic elastic tape 102 (in the z axis), or other structures for stimulating blood flow, guiding liquidated medications, and channeling perspiration.

In one embodiment, the medicated dots may each include a micro needle or nano tube delivery system for further delivering the medications directly into the patient's body. The micro needle/duct may be small enough to not irritate the skin during utilization. In another embodiment, the micro needles/duct may be configured to be absorbed by the skin once the medications are delivered or during the delivery process. For example, micro structures may be embedded within the skin during contact to deliver the medications.

In one embodiment, the medication is configured to dissolve into the users skin to apply the medication. For example, the therapeutic elastic tape may include medication that is integrated in a solvent that dissolves, evaporates or breaks down in response to perspiration, heat, pressure, friction, or so forth.

The pattern 110 of the therapeutic elastic tape 104 may utilize medicated strips running along a length of the therapeutic elastic tape 104. The medicated strips may run longitudinally (as shown), laterally, diagonally, or utilizing a combination of patterns.

The pattern 112 shows one embodiment of a varied pattern of horizontal and diagonal components that may be utilized in the therapeutic elastic tape 106. The patterns 102, 104, and 106 may also utilize any number of structured or random patterns. The overall shape and configuration of the therapeutic elastic tape 102, 104, and 106 may vary as is further shown in the illustrative embodiments. In one embodiment, the therapeutic elastic tape may include an elastic core layer configured to deform or stretch based on forces (e.g. muscle expansion) and then return to the original size. The elastic core layer

FIG. 2 is a spreadsheet 200 of medications for therapeutic elastic tape in accordance with an illustrative embodiment. As shown the therapeutic elastic tape may include any number of medication, vitamins, electrolytes, drugs, or other components either singly or in combination. In one embodiment, the medicines are combined for diffusion through the skin. The medications may be integrated or combined with a delivery agent. For example, the medications may be bound to Dimethyl sulfoxide (DMSO) or other appropriate delivery/transport agents in order to deliver drugs through the skin and membranes into muscle and tissue.

The spreadsheet 200 represents various medications for utilization with the therapeutic elastic tape. As shown, the therapeutic elastic tape may be especially configured for training, competition, and recovery. The therapeutic elastic tape may include over the counter (OTC) medications or prescription (RX) medications. As a result, the therapeutic elastic tape may be self-applied by a user or applied by a medical professional including doctors, nurses, physician's assistants, athletic trainers, and so forth.

The therapeutic elastic tape and corresponding medications may be utilized to treat strains, spasms, pain, aches, swelling/edema, fascitis, bursitis, and so forth. The therapeutic elastic tape may be particularly useful for treating muscles, tendons, ligaments, joints, fascia, and bursa (see Roman Numerals).

In one embodiment, the medications include DMSO, Sombra, electrolytes, sodium (NaCl), Potassium K+, Magnesium (Mg), vitamins (C, B1, B2, D, E, Niacin, B6, B12, Calcium, Iron, Magnesium, and Zinc), caffeine, menthol, camphor, ibuprofen, hydrocortisone, and lanolin. Examples of prescription drugs that may be utilized include DMSO, Testosterone, Betamethasone, Benzocaine, Capsaicin, Diphenhydramine, Lidocaine, Marcaine, Tetracaine, Ketaprofen, Voltaren, and Cyclobenzaprine. However, any number of drugs or dietary supplements, herbal products suitable for treating a user may be utilized; including but not limited to: stimulants, amino acids, vasodilators/vasoconstrictors and recovery agents. The therapeutic elastic tape may also be utilized to deliver medications that may be resisted by the user during direct delivery (e.g. orally, or through other body orifices).

The medications of spreadsheet 200 may be applied in any number of combinations based on the needs of the user. The therapeutic elastic tape may be prescribed by a medical professional in the same way other medications are prescribed to users or patients. In one embodiment, the distinct medications, herbs, vitamins, supplements, or chemicals may be separately adhered or integrated with the therapeutic elastic tape. For example, the medications may be deposited in gel strips, medicated dots, or other patterns that run along the therapeutic elastic tape. The separation of the medications may prevent cross-contamination, negative reactions, dilution, or ineffectiveness due to mixing when applied, dissolved, or absorbed by the user. In one embodiment, mechanical components, such as ridges, separators, or so forth may separate the distinct medications.

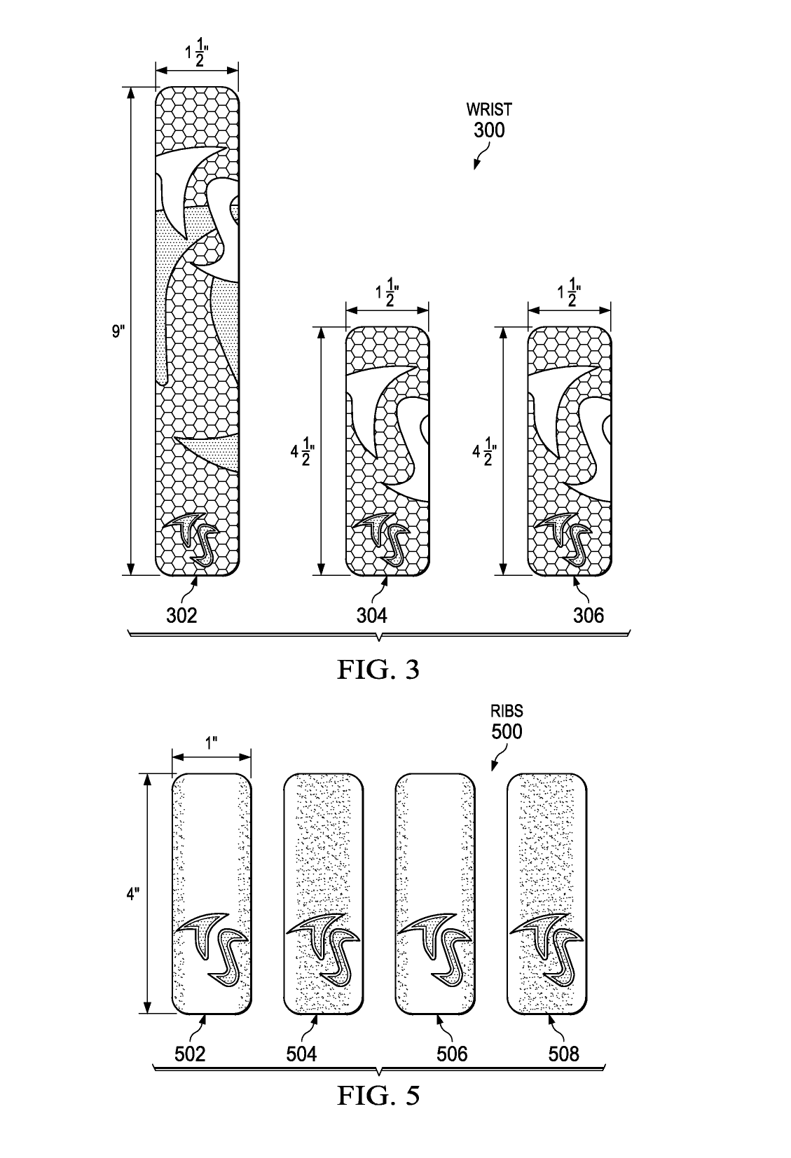

As shown in FIGS. 3-18, the therapeutic elastic tapes may include multiple distinct tape sections or perforations for separating sections or portions of the therapeutic elastic tape as shown. As a result, the therapeutic elastic tape may be properly distributed for the targeted area or appendage. As previously mentioned, the therapeutic elastic tape may also include a backing layer that is removed before application of the tape. In other embodiments, the therapeutic elastic tape may be stored in independent vacuum sealed packages. The perforations may further facilitate the placement of the therapeutic elastic tape by allowing the user to best secure the distinct portions of the therapeutic elastic tape. The measurements provided for the different embodiments of the therapeutic elastic tape may be increased or decreased as needed based on the type of user (e.g. male, female, child, adult, etc.) The therapeutic elastic tape may include any number of segments or distinct pieces that make up a unit or treatment kit. Each segment may additionally have sections that may be physically separated during positioning and application. The sections may be separated from each other or the body of the therapeutic elastic tape by perforations or other mechanical weaknesses.

FIG. 3 is a pictorial representation of therapeutic elastic tape 300 for a wrist of a user in accordance with an illustrative embodiment. In one embodiment, segment 302 is 9″ long and 1.5″ wide. Segments 304 and 306 may be 4.5″ long and 1.5″ wide.

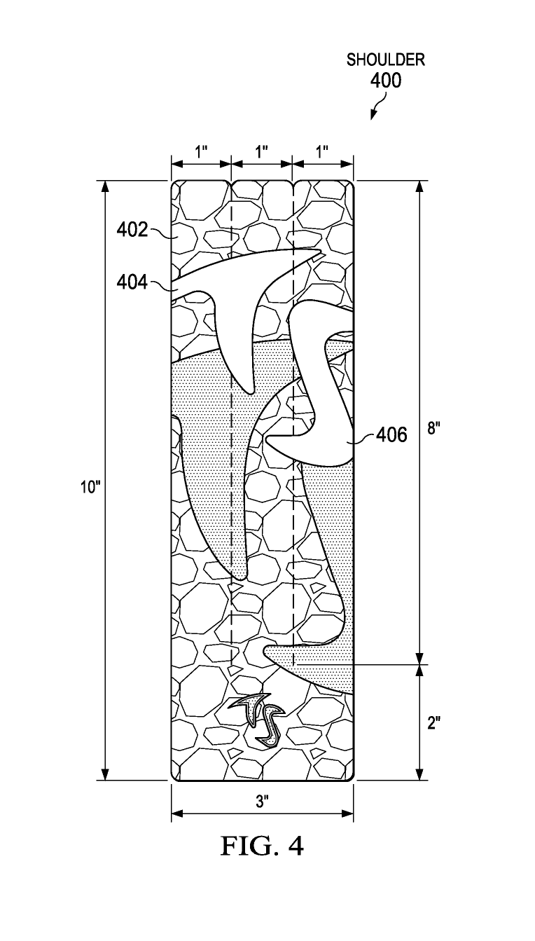

FIG. 4 is a pictorial representation of therapeutic elastic tape 400 for a shoulder of a user in accordance with an illustrative embodiment. In one embodiment, the therapeutic elastic tape 400 may be 10″ long and 3″ wide. A portion of the therapeutic elastic tape may be separable to form sections 402, 404, and 406 utilizing perforations into 1″ wide segments that are 8″ long and extend longitudinally along the therapeutic elastic tape. The sections 402, 404 and 406 may be separated to secure and treat an expanded portion of the user's shoulder.

FIG. 5 is a pictorial representation of therapeutic elastic tape 500 for ribs of a user in accordance with an illustrative embodiment. The therapeutic elastic tape 500 may be packaged to include segments 502, 504, 506, and 508. Each of the segments 502, 504, 506, and 508 may be 4″ long and 1″ wide. The segments 502, 504, 506, and 508 may be positioned laterally or longitudinally along a portion of a user's ribs.

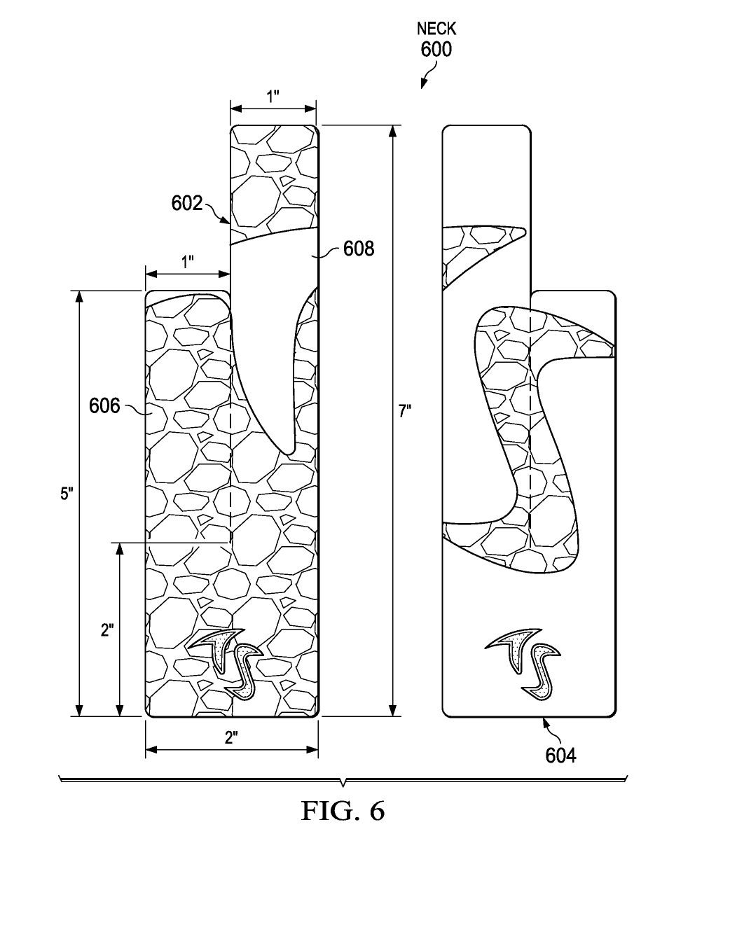

FIG. 6 is a pictorial representation of therapeutic elastic tape 600 for a neck of a user in accordance with an illustrative embodiment. In one embodiment, the therapeutic elastic tape 600 may include segments 602 and 604. Each of the segments 602 and 604 may have distinct sections for positioning on the user's neck. The segments 602 and 604 are 7″ long at the longest point of section 606 and 5″ long at the shortest point of section 608. Each section 606 and 608 is 1″ wide and the segments 602 and 604 are 2″ wide. The perforation between sections 606 and 608 may extend to 2″ from the bottom most portion of the therapeutic elastic tape 600.

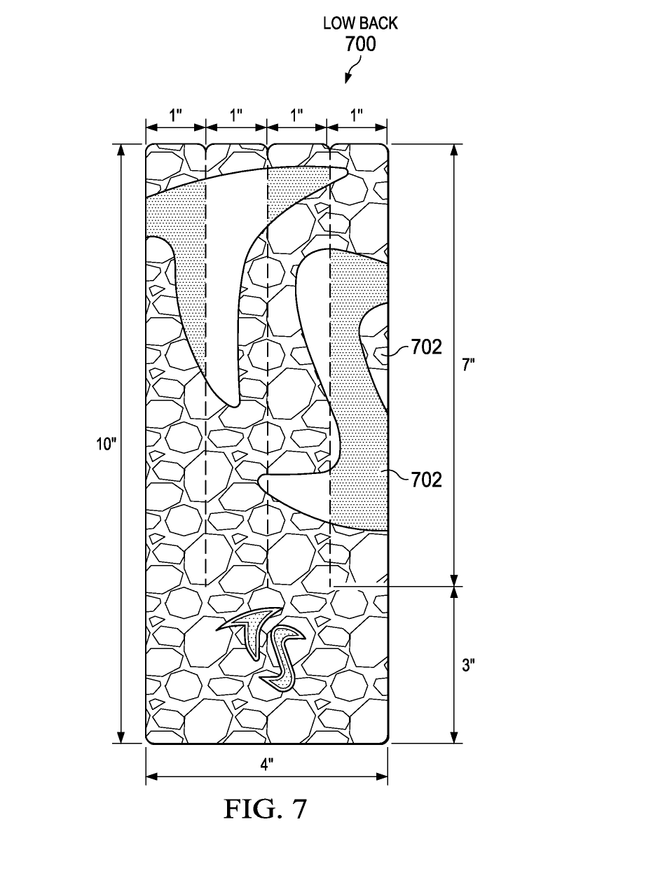

FIG. 7 is a pictorial representation of therapeutic elastic tape 700 for a lower back of a user in accordance with an illustrative embodiment. The therapeutic elastic tape may be 10″ long overall and 4″ wide. In one embodiment, the therapeutic elastic tape includes four sections 702. Each of the sections 702 may be 1″ wide and 7″ long. The perforation separation the sections 702 may extend to 3″ from the bottommost portion of the therapeutic elastic tape. The increased number of sections 702 may allow for expanded coverage of the lower back.

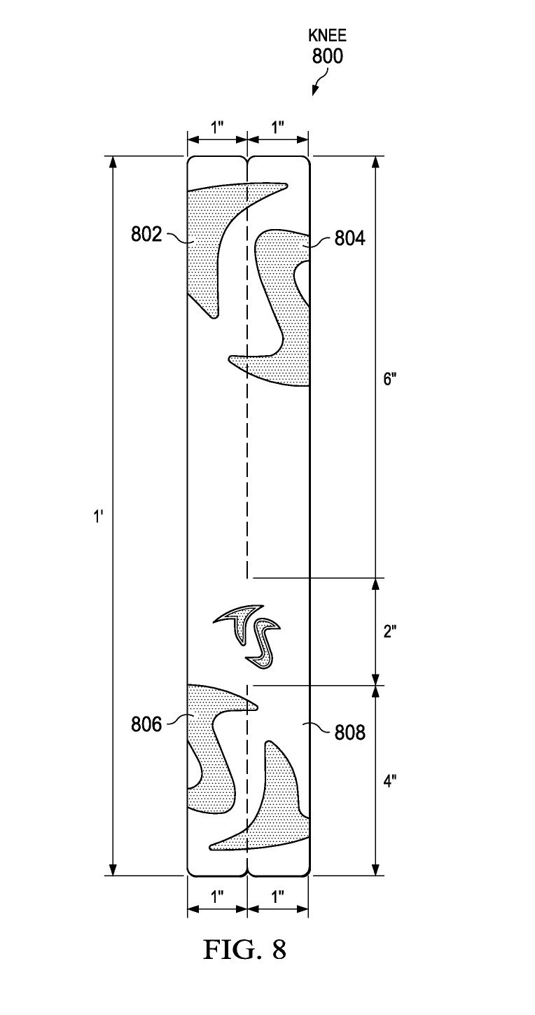

FIG. 8 is a pictorial representation of therapeutic elastic tape 800 for a knee of a user in accordance with an illustrative embodiment. The therapeutic elastic tape 800 may include sections 802, 804, 806, and 808. The therapeutic elastic tape 800 may be 12″ long overall and 2″ wide. Each of the sections may be 1″ wide and sections 802 and 804 may be 6″ long and sections 806 and 802 may be 4″ long.

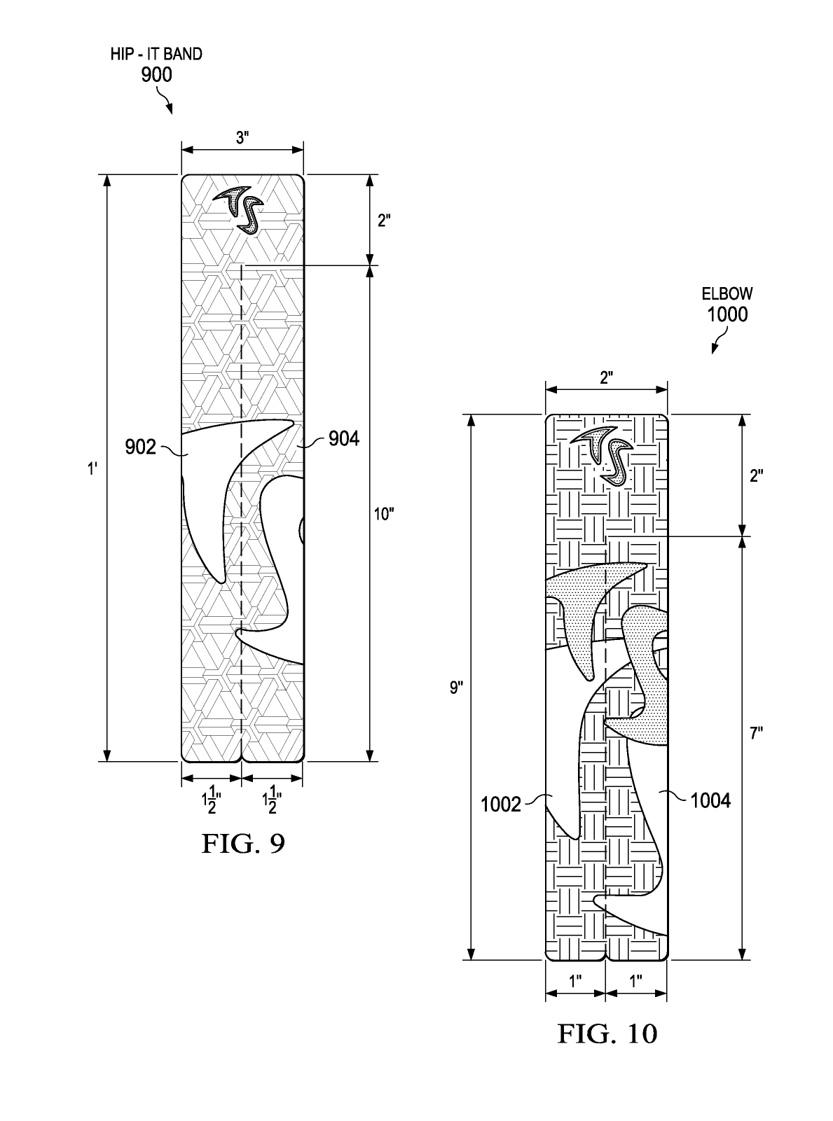

FIG. 9 is a pictorial representation of therapeutic elastic tape 900 for a hip of a user in accordance with an illustrative embodiment. The therapeutic elastic tape 900 may be 12″ long overall and 3″ wide. The therapeutic elastic tape 900 may include sections 902 and 904. Sections 902 and 904 may be 1.5″ wide and 10″ long. The perforation between the sections 902 and 904 may extend 10″ from the bottommost portion of the therapeutic elastic tape 900.

FIG. 10 is a pictorial representation of therapeutic elastic tape 1000 for an elbow of a user in accordance with an illustrative embodiment. The therapeutic elastic tape 1000 may be 9″ long overall and 2″ wide. The therapeutic elastic tape 1000 may include sections 1002 and 1004. Sections 1002 and 1004 may be 1″ wide and 7″ long. The perforation between the sections 902 and 904 may extend 7″ from the bottommost portion of the therapeutic elastic tape 1000.

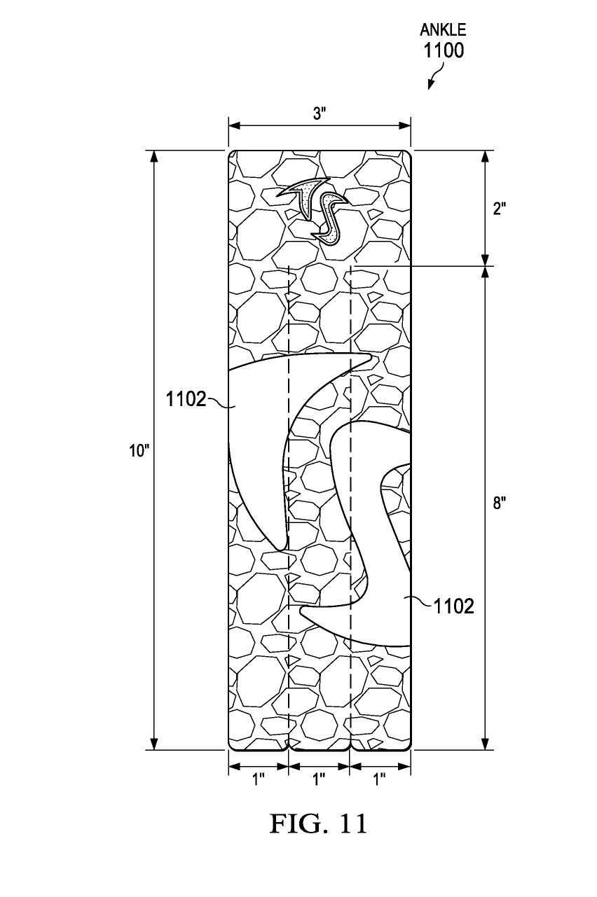

FIG. 11 is a pictorial representation of therapeutic elastic tape 1100 for an ankle of a user in accordance with an illustrative embodiment. The therapeutic elastic tape 1100 may be 10″ long overall and 3″ wide. The therapeutic elastic tape 1100 may include three sections 1102 each of which is 1″ wide and extends 8″. Similarly, the perforations may extend 8″.

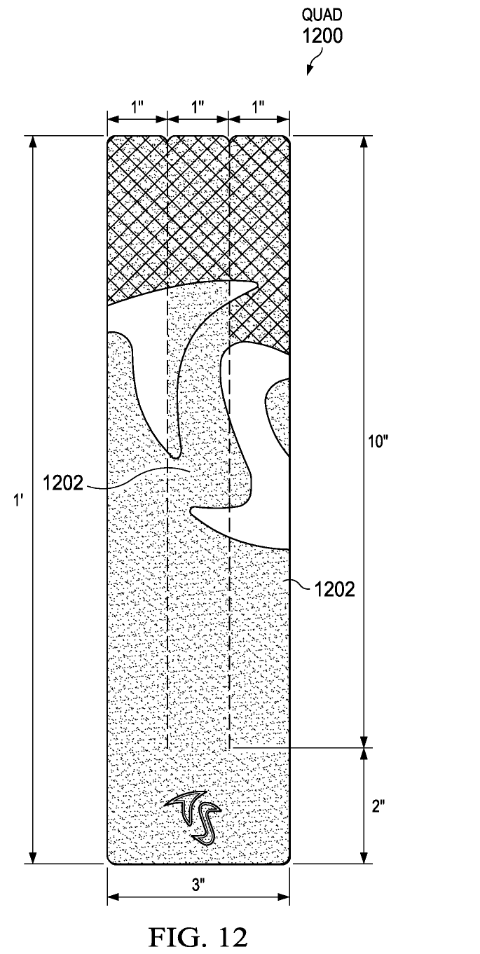

FIG. 12 is a pictorial representation of therapeutic elastic tape 1200 for a quadricep of a user in accordance with an illustrative embodiment. The therapeutic elastic tape 1200 may be 12″ long overall and 3″ wide. The therapeutic elastic tape 1200 may include three sections 1202 each of which is 1″ wide and extends 10″. Similarly, the perforations may extend 10″.

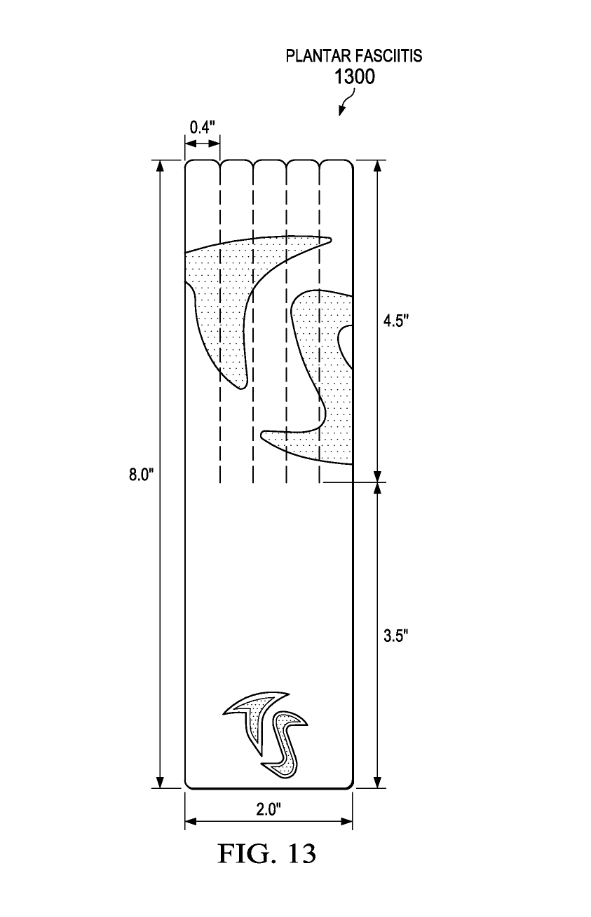

FIG. 13 is a pictorial representation of therapeutic elastic tape 1300 for treating plantar fasciitis of a user in accordance with an illustrative embodiment. The therapeutic elastic tape 1300 may be 8″ long and 2″ wide. The therapeutic elastic tape 1300 may include five sections 1302 each of which is 0.4″ wide and 4.5″ long. Similarly, the perforations may extend 4.5″.

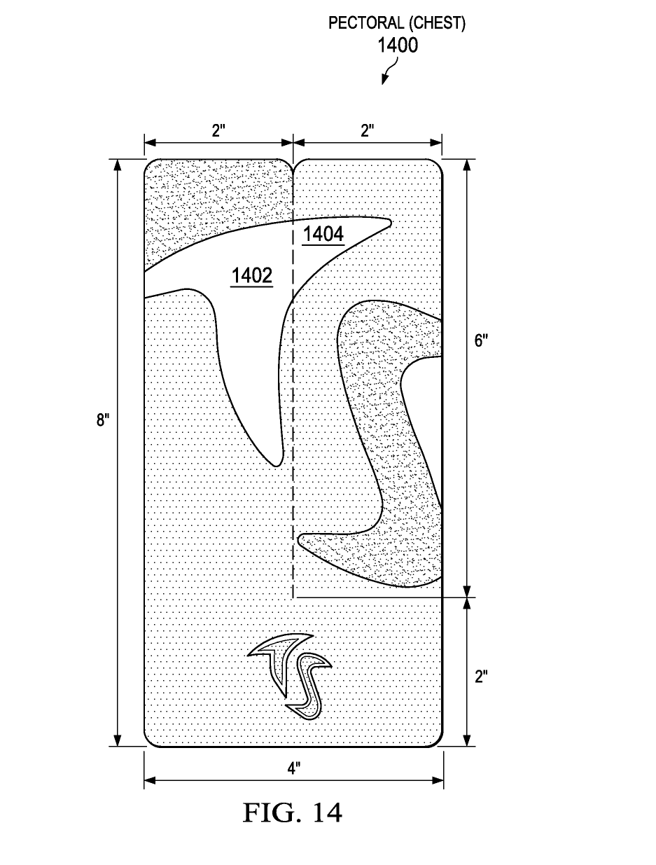

FIG. 14 is a pictorial representation of therapeutic elastic tape 1400 for treating a chest of a user in accordance with an illustrative embodiment. The therapeutic elastic tape 1400 may be 8″ long overall and 4″ wide. The therapeutic elastic tape 1400 may include sections 1402 and 1404. The sections may each be 2″ wide and 6″ long. Similarly, the perforations may extend 6″.

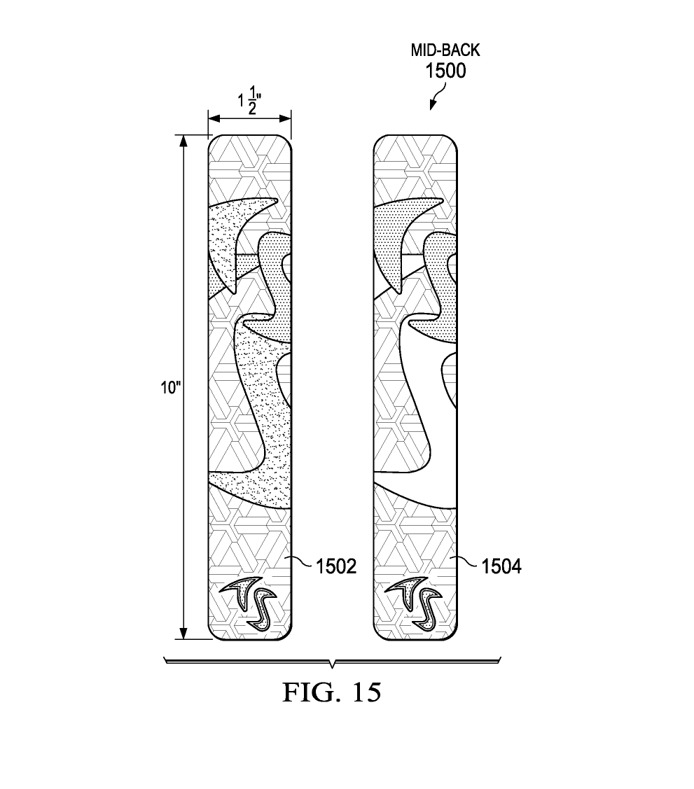

FIG. 15 is a pictorial representation of therapeutic elastic tape 1500 for treating a mid-back of a user in accordance with an illustrative embodiment. The therapeutic elastic tape 1500 may include segments 1502 and 1504. Each segment 1502 and 1504 may be 10″ long overall and 1.5″ wide.

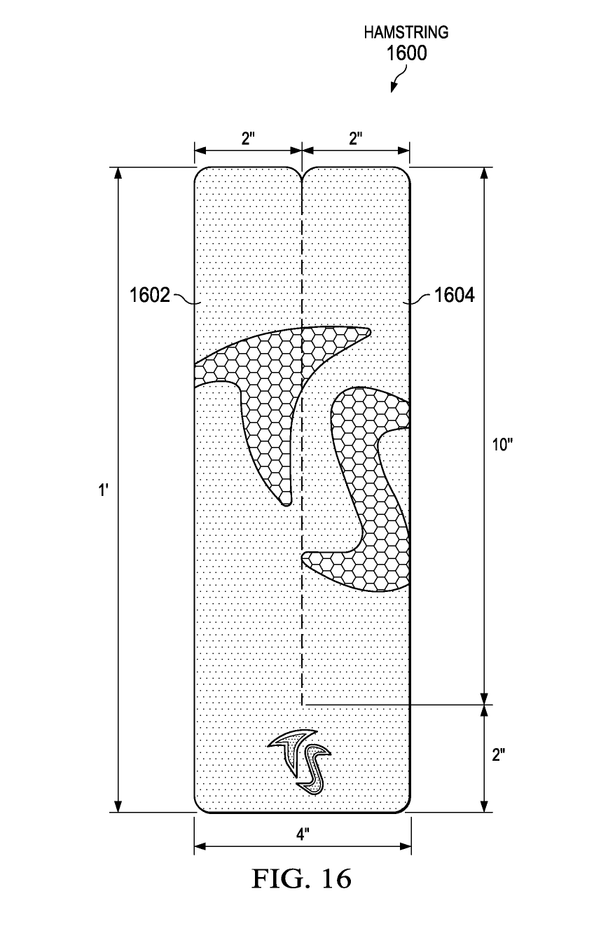

FIG. 16 is a pictorial representation of therapeutic elastic tape for treating a hamstring of a user in accordance with an illustrative embodiment. The therapeutic elastic tape 1600 may be 10″ long and 4″ wide. The therapeutic elastic tape 1600 may include sections 1602 and 1604. The sections may each be 2″ wide and 10″ long. Similarly, the perforations may extend 10″.

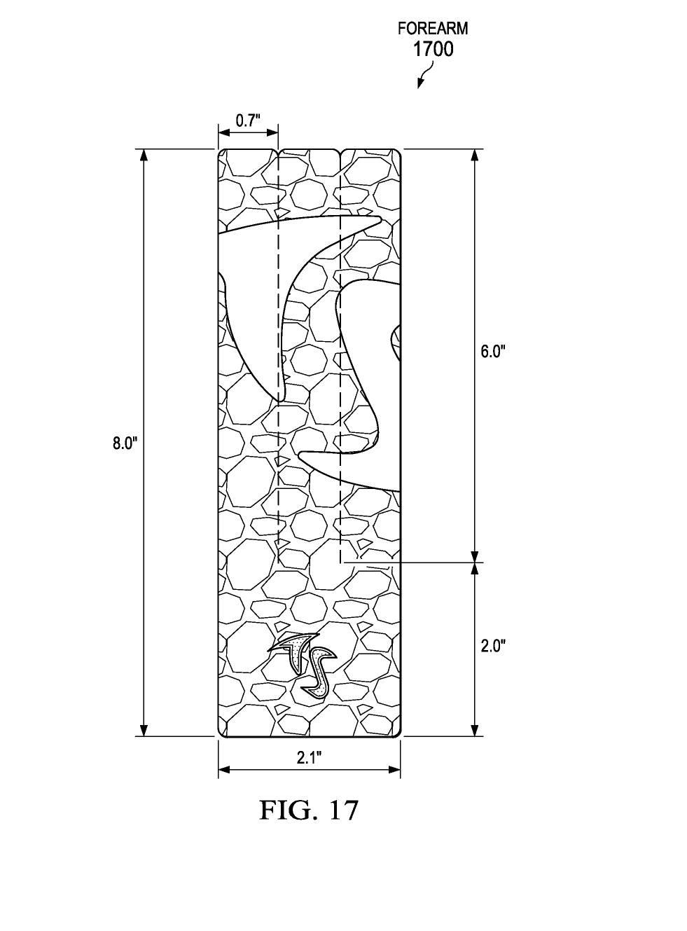

FIG. 17 is a pictorial representation of therapeutic elastic tape 1700 for treating a forearm of a user in accordance with an illustrative embodiment. The therapeutic elastic tape 1700 may be 8″ long overall and 2.1″ wide. The therapeutic elastic tape 1700 may include three sections 1702. Each of the sections 1702 may be 0.7″ wide and 6″ long. Similarly, the perforations may extend 6″.

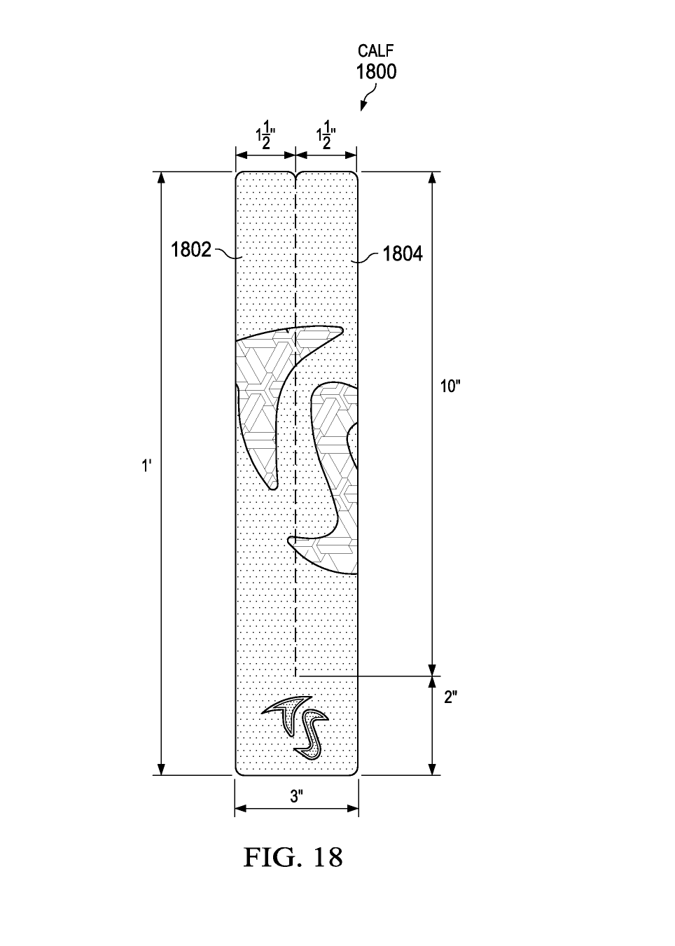

FIG. 18 is a pictorial representation of therapeutic elastic tape 1800 for treating a calf of a user in accordance with an illustrative embodiment. The therapeutic elastic tape 1800 may be 12″ long overall and 3″ wide. The therapeutic elastic tape may include sections 1802 and 1804. Each of the sections 1802 and 1804 may be 1.5″ wide and 10″ long. Similarly, the perforations may extend 10″.

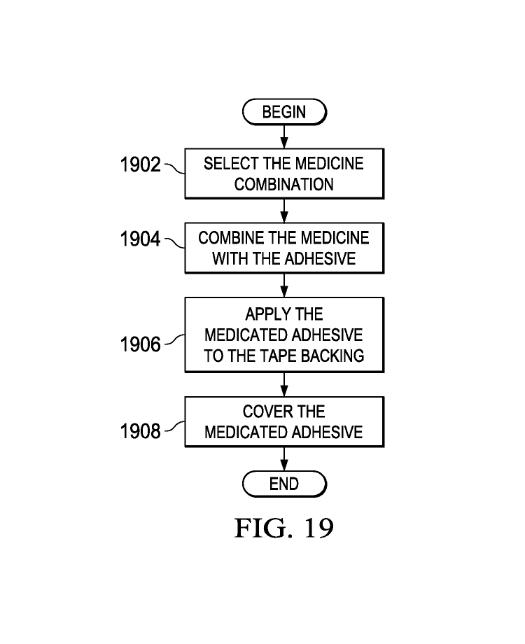

FIG. 19 is a flowchart of a process for manufacturing therapeutic elastic tape in accordance with an illustrative embodiment. The process of FIG. 19 may be implemented for rolls of therapeutic elastic tape. The therapeutic elastic tape may be manufactured in rolls. In one embodiment, the process of FIG. 19 may be implemented utilizing a number of drums, rollers, sprayers, immersion baths, and utilizing a method, composition, and structure as are described in U.S. Pat. Nos. 3,523,859, 5,397,298, and 5,861,348 which are hereby incorporated by reference. In one embodiment, the structure of the therapeutic elastic tape may be manufactured and assembled before the process of FIG. 19 is implemented.

The process may begin by selecting a medicine combination (step 1902). The medicine combination may include any number of drugs, vitamins, supplements, or so forth as are herein described. A single medication or multiple medications (e.g. a delivery drug and a pain killer)

Next, the system combines the medicine with the adhesive (step 1904). In one embodiment, the adhesive is an acrylic based synthetic resin. The medicine may be combined with the adhesive prior to application or integrated with the medicated adhesive tape or at the time of application.

Next, the system applies the medicated adhesive to the tape backing (step 1906). In one embodiment, the tape backing is the portion of the therapeutic elastic tape that is designated for contact with the body of the user. The entire tape backing may be configured to receive the medicated adhesive. In another embodiment, a pattern of medicated adhesive may be applied to the tape.

In one embodiment, the tape is rolled with a dispenser coating a back side of the tape with the medicated adhesive. In another embodiment, the tape may be dipped in an adhesive. In another embodiment, the tape may be sprayed with the medicated adhesive. In yet another embodiment, the tape may be injected with the medicated adhesive. For example, needles may inject the medicated adhesive into the first 0.5 mm or necessary effective depth of the therapeutic elastic tape.

In another embodiment, the adhesive and the medications may be applied separately or in distinct layers. The adhesive may be applied in a first pattern and the medicine in a second pattern.

Next, the system covers the medicated adhesive (step 1908). In one embodiment, the therapeutic elastic tape is covered by a peel-off backing or cover. For example, a plastic cover may be utilized to cover the tape backing preventing inadvertent adhesion before the therapeutic elastic tape is applied to the designated user. In another embodiment, the therapeutic elastic tape may be wrapped onto itself in rolls. The therapeutic elastic tape may be manufactured in rolls or in specific sizes and shapes as is shown in FIGS. 3-11. The therapeutic elastic tape may be packaged in sleeves, bags, wrapping, or otherwise stored for shipping before utilization.

The therapeutic elastic tape is a consumer-product, allowing ease of accessibility to the general public, teams, institutions, organizations and entities that may benefit from the use of therapeutic elastic tape. The aforementioned therapeutic elastic tape system (tape fabric, active adhesive and treatments within the adhesive) may be manufactured separately or jointly. The therapeutic elastic tape may be achieved by coalescing active tape-adhesive with over-the-counter anti-inflammatories, prescription medications, anesthetics, analgesics and counter-irritants with an externally-placed fastening system primarily functioning as an adaptable/flexible bio-tape.

In one embodiment, the therapeutic elastic tape may be made available in four different product lines: 1. Practice/training line; 2. Competition/performance line; 3. Recovery or post-competition line; and 4. Prescription line. 5. Wellness line

In one embodiment, the therapeutic elastic tape for each body region to be treated may be pre-designed, fabricated and sold in individual packages. The therapeutic elastic tape may be available in pediatric through adult sizes (e.g. pediatric sizes: Youth small, medium and large. Adult sizes: small, medium and large for each region of tape application). The chosen size and area to be treated may determine the length and width of each therapeutic elastic tape application kit. In one embodiment, physical composition of therapeutic elastic tape including the outer-surface of the tape may include a cotton-fiber blend with innate stretching ability.

This outer surface of the tape (or visible side once applied) may be available in numerous, brilliant designs, and colors. All visible and non-visible colors of the spectrum may be available. Custom colors may also be available (e.g. a school team may order bulk quantities of their school colors, which may represent two or more color combinations). The outer surface may also include any number of marks, phrases, slogans, instructions, logos, text, graphics or so forth that are useful for utilization or desirable for aesthetic purposes. In one embodiment, the outer surface may be dark for attracting sunlight/warmth to activate the medications for delivery. For example, the medications may be delivered once the temperature of the therapeutic elastic tape reaches a threshold temperature. For example, the threshold temperature may be 104°. In another embodiment, the outer surface of the tape may be light-colored or reflective to prevent the therapeutic elastic tape from absorbing solar or other energy from outside sources. Any number of color schemes or reflective materials such as metal or other materials may be utilized in the therapeutic elastic tape.

The outer surface of the therapeutic elastic tape may include visibly reveal lines of demarcation, markers, indicators, or instructions which may represent and overlap the medicated treatment zone on the inner-surface of the tape (or the side of the tape directly contacting the skin). The therapeutic elastic tape may include external markings and instructions indicating how and when to apply the specific type of therapeutic elastic tape. The therapeutic elastic tape may also include markings indicating the type of medications being delivered for reference by a nurse, physicians assistant, doctor or other medical professional or personnel in a personal or commercial setting (e.g. home, nursing home, doctors office, hospital). The anchors or ends of the tape on the inner surface may be medicated.

The inner-surface of the tape may include an acrylic-based adhesive impregnated with the aforementioned over-the counter class of medicated treatments, prescription treatments, vitamins, etc. The inner-surface of the therapeutic elastic tape may be contiguous with a separate paper/release liner or plastic based backing to preserve the quality and integrity of the medicated adhesive. (This barrier or backing is removed prior to application). Application may be performed in a simple sequence: 1. The therapeutic elastic tape may be separated from the paper barrier, backing, or package; 2. The primary anchor of the therapeutic elastic tape may be applied; 3. The therapeutic elastic tape may be placed on a minimal stretch; covering the area to be treated and the secondary anchor/s applied; and 4. The therapeutic elastic tape may be activated by gently rubbing its outer surface, applying heat, infrared light, or other electronic signals, user motion, or other internal or external stimuli. The therapeutic elastic tape may serve several concurrent functions: Firstly, the therapeutic elastic tape surrounds and stabilizes the automated percutaneous treatment distribution system and second the therapeutic elastic tape facilitates or inhibits musculoskeletal processes determined by the manner of placement of the therapeutic elastic tape and adhesive that may be utilized. In one embodiment, the packaged therapeutic elastic tape may be a single use disposable product. In another embodiment, the therapeutic elastic tape may be sprayed, soaked, or dipped in additional medications for reapplication. For example, new adhesive ends may be secured to the therapeutic elastic tape to reapply one or more segments.

The therapeutic elastic tape may be applied utilizing any number of shapes or techniques. In one embodiment, the various segments may be positioned in overlapping or non-overlapping positions. In one embodiment, and “I” shape is utilized for small or linear places. A “Y” shape may be utilized for larger muscles. And “X” shape may be used for large and long muscles. Additionally, the distinct sections of the therapeutic elastic tape may be connected in name nodal configuration in which the sections spread out from a node point or base section of the therapeutic elastic tape as is shown in various embodiments in FIGS. 3-18. For example, the sections may be utilized to spider out over a section of the user's body.

The previous detailed description is of a small number of embodiments for implementing the invention and is not intended to be limiting in scope. The following claims set forth a number of the embodiments of the invention disclosed with greater particularity.

What is claimed:

1. Therapeutic elastic tape, comprising:
an elastic fabric having a back side and a face side;
an adhesive material applied to the elastic fabric; and
a medication applied to at least the back side of the elastic fabric for diffusion to a portion of a body of the user during utilization.

2. The therapeutic elastic tape according to claim 1, wherein the medication is integrated with the adhesive material.

3. The therapeutic elastic tape according to claim 1, wherein the medication is applied in a pattern.

4. The therapeutic elastic tape according to claim 1, wherein the medication and adhesive material do not overlap.

5. The therapeutic elastic tape according to claim 1, wherein the medication and adhesive material overlap.

6. The therapeutic elastic tape according to claim 1, wherein the therapeutic elastic tape further comprises:
an impermeable layer separating the adhesive material and the medication from the face side.

7. The therapeutic elastic tape according to claim 1, wherein the medication include one or more of over the counter medications, prescription medications, vitamins, herbal and dietary supplements, and electrolytes.

8. The therapeutic elastic tape according to claim 1, wherein the elastic fabric and the adhesive material are kinesio tape.

9. The therapeutic elastic tape according to claim 1, wherein the medication is configured for time-based delivery.

10. The therapeutic elastic tape according to claim 1, wherein the medication is heat and motion activated for delivery to the user.

11. The therapeutic elastic tape according to claim 1, wherein the face sides includes markers indicating where to apply the therapeutic elastic tape to the portion of the body of the user.

12. A method of manufacturing therapeutic elastic tape, comprising:
combining a fabric having a back side and a face side;
applying an adhesive material to the back side of the fabric; and
applying a medication to at least the back side of the fabric for absorption by a user when the therapeutic elastic tape is applied.

13. The method of manufacturing according to claim 12, wherein the applying comprises:
infusing the medication in a delivery layer.

14. The method of manufacturing according to claim 12, wherein the adhesive material and the medication are combined.

15. The method of manufacturing according to claim 12, wherein the back side further comprises:
a delivery layer configured to receive the adhesive material and the medication.

16. The method of manufacturing according to claim 14, wherein the adhesive material is an acrylic-based adhesive.

17. Therapeutic elastic tape, comprising:
an elastic fabric having a back side and a face side;
an adhesive material applied to the back side of the elastic fabric, wherein the adhesive material includes a medication for absorption by into a body of a user when the therapeutic elastic tape is applied to the user.

18. The therapeutic elastic tape according to claim 17, wherein the back side further comprises:
a delivery layer configured to receive the adhesive material.

19. The therapeutic elastic tape according to claim 17, wherein the medication includes a delivery agent and one or more of a prescription drug, over the counter drug, herb, or vitamin.

20. The therapeutic elastic tape according to claim 17, wherein the adhesive material includes one or more delivery layers, wherein the one or more delivery layers include an adhesive layer for attaching the therapeutic elastic tape to a body of a user, and medication for absorption by the body of the user when the therapeutic elastic tape is applied to the user.
